(19) United States
(12) Patent Application Publication
(10) Pub. No.: US 2021/0398649 A1
(43) Pub. Date: Dec. 23, 2021

(54) MULTI-DIMENSIONAL BIG HEALTH GUIDANCE SYSTEM BASED ON TRADITIONAL CHINESE MEDICINE THEORY

(71) Applicant: XIAMEN ZHI HUI QUAN TECHNOLOGY CO. LTD, Xiamen (CN)

(72) Inventor: Ji-Li CHUNG, Xiamen (CN)

(21) Appl. No.: 17/292,153

(22) PCT Filed: Nov. 11, 2019
(86) PCT No.: PCT/CN2019/117026

§ 371 Date: May 7, 2021

(30) Foreign Application Priority Data
Nov. 12, 2018 (CN) 201811336970.3

(51) Int. Cl. G16H 20/90; G16H 40/67; G16H 10/60; G16H 50/70

(52) U.S. Cl. CPC G16H 20/90; G16H 40/67; G06K 9/00221; G16H 50/70; G16H 10/60

(57) ABSTRACT

A multi-dimensional big health guidance system based on a traditional Chinese medicine theory, and a multi-dimensional big health guidance method based on a traditional Chinese medicine theory are provided. An information acquisition unit is used to collect user information. An information analysis unit is used to analyze the user information and perform a user portrait. A guidance scheme output unit is used to perform relevance matching on the user portrait and the material data stored in an information storage unit from multiple dimensions, so as to output a guidance scheme matching the user information. The guidance scheme matching is performed on the user from multiple dimensions, and finally, more accurate and precise health guidance is provided to the user.

This application is the 35 U.S.C. § 371 national stage of PCT application PCT/CN2019/117026, filed Nov. 11, 2019, which claims the benefit of People's Republic of China application serial No. 201811336970.3, filed Nov. 12, 2018, the disclosure of which is hereby incorporated by reference.

### BACKGROUND OF THE INVENTION

### Field of the Invention

The invention relates to a field of software program, in particular to a multi-dimensional big health guidance system based on a traditional Chinese medicine theory.

### Description of the Related Art

The big health is a global idea which is developed according to age development and social demands, and the global idea of big health is focused on people's life and attracting people's living and illness, and a self-health management is advocated for various dangerous factors and misunderstandings that affect our health. The pursuit of the big health is not only the body health of the individual, but also comprises the full health of mental, psychology, physiology, society and the like.

A healthy lifestyle is advocated by the big health, not only is “disease treatment”, but also to prevent the illness, eliminate the illness and be healthy, improvement of physical quality, reduction of pain, health care, health management and health maintenance. The big health guidance helps people to shift from exhausting their health and fighting the illness to a new health model that cares for their health and prevents the illness.

Health management is a method capable of actively and effectively grasping and maintaining health, comprehensively investigating, analyzing, evaluating, monitoring and predicting a personal health state, and taking an intervention measure for health risk factors so as to reduce or eliminate risk factors and guarantee his/her good health state.

At present, most of the existing health guidance programs on the market are one-dimensional health guidance, such as providing anti-hypertensive or low-sugar diet guidance for people with hypertension or diabetes. However, according to the traditional Chinese medicine theory system, human beings, as an organism of essence, qi, and spirit, are affected by various internal and external factors, and human viscera, meridians and blood saliva change continuously over time. This type of one-dimensional health guidance is not certainly instructive for a specific situation of various internal and external factors, and would not be able to satisfy people's ultimate pursuit of big health.

### SUMMARY OF THE INVENTION

In order to solve the above problems, the invention provides a multi-dimensional big health guidance system based on the traditional Chinese medicine theory, which can comprehensively consider the influence factors of a plurality of dimensions, and provides a more accurate health guidance scheme for people.

In an embodiment of the invention, a multi-dimensional big health guidance system based on a traditional Chinese medicine theory is provided, which comprises an information acquisition unit for collecting user information; an information analysis unit for analyzing the user information and performing a user portrait; an information storage unit for storing material data related to user's basic necessities; and a guidance scheme output unit for performing relevance matching on the user portrait and the material data stored in the information storage unit from multiple dimensions, so as to output a guidance scheme matching the user information.

In an embodiment of the invention, the information acquisition unit includes one or more of a data input module, an internet information acquisition module, a wearable device information receiving module, a face recognition module and a voice semantic recognition module.

In an embodiment of the invention, the user information includes one or more of basic information, disease information, physique information, physiological information, habitual information, behavior information, emotional information, and environmental information.

In an embodiment of the invention, the material data includes one or more of food material data, a medicated diet formula, an exercise method, a meridian massage, a bath method, a tea drink, and an audio-visual material.

In an embodiment of the invention, the user portrait includes multi-dimensional information data.

In an embodiment of the invention, the information storage unit further stores correlation data between the material data and the user information data, and the correlation data is based on the traditional Chinese medicine theory.

In an embodiment of the invention, the guidance scheme includes a catering scheme, a sports scheme, a leisure scheme, and a beauty care scheme. The catering scheme is applied to a meal ordering application (APP); the sports scheme or the leisure scheme is applied to a sports or a health class APP; and the beauty care scheme is applied to a beauty treatment APP.

In an embodiment of the invention, a multi-dimensional big health guidance method based on a traditional Chinese medicine theory is provided, which comprises the following steps: S1, collecting user information, S2, analyzing the user information and performing a user portrait; S3, evaluating material data stored in the information storage unit according to the user portrait; S4, outputting a guidance scheme suitable for the user portrait.

In an embodiment of the invention, the specific method applied in step S1 includes one or more of a user input, an image scanning, a wearable device information receiving, an internet access, a face recognition and a voice semantic recognition.

In an embodiment of the invention, the user information in step S2 is converted into information data according to a plurality of dimensions, and the information data is three-dimensionally structured to form the user portrait.

In an embodiment of the invention, the step S3 further includes: analyzing the information data from a first dimension to screen a first dimensional guidance scheme matching first dimensional information data from the information storage unit; and then analyzing the information data from a second dimension to screen a two-dimensional guidance scheme matching the second dimensional information data from the first dimensional guidance scheme.

In an embodiment of the invention, the step S3 further includes analyzing the information data from a third dimension to screen a three-dimensional guidance scheme matching third dimensional information data from the two-dimensional guidance scheme.

In an embodiment of the invention, the step S3 further includes: analyzing the information data from first dimension to screen a first dimensional guidance scheme matching the first dimensional information data from the information storage unit; and synchronously analyzing the information data from a second dimension to screen a second dimensional guidance scheme matching the second dimensional information data from the information storage unit.

In an embodiment of the invention, the step S3 further includes: analyzing first dimensional guidance scheme and second dimensional guidance scheme and screening an intersection set as a two-dimensional guidance scheme.

In an embodiment of the invention, the step S3 further includes: synchronizing the information data from a third dimension to screen a third dimensional guidance scheme matching the third dimensional information data from the information storage unit.

In one embodiment of the invention, first dimension and second dimension are one of personal factors and environmental factors, respectively.

In one embodiment of the invention, first dimension and second dimension are respectively different aspects of personal factors.

For the multi-dimensional big health guidance method based on the traditional Chinese medicine theory, the user information is split into information data of multiple dimensions, a user portrait is performed, and a guidance scheme matching on the user from multiple dimensions can be accurately and precisely carried out, and thus the health guidance is finally provided suitable for the user, and the multi-dimensional big health guidance method is beneficial to better popularization of the big health idea and health industry.

Other objects, features, and advantages of the invention will become apparent from the following detailed description of the preferred but non-limiting embodiments. The following description is made with reference to the accompanying drawings.

### BRIEF DESCRIPTION OF THE DRAWINGS

FIG. 1 is a schematic diagram of a multi-dimensional big health guidance system based on a traditional Chinese medicine theory.

FIG. 2 is a schematic diagram of a multi-dimensional big health guidance method based on a traditional Chinese medicine theory.

FIG. 3 is a schematic diagram of a multi-dimensional hierarchical matching method according to an embodiment of the invention.

FIG. 4 is a schematic diagram of a multi-dimensional synchronous matching method according to an embodiment of the present invention.

### DETAILED DESCRIPTION OF THE INVENTION

To make the objectives, technical solutions and advantages of the present invention clearer, the present invention will be further described in detail with reference to the accompanying drawings and embodiments. It should be understood that the specific embodiments described herein are merely used to explain the present invention and are not intended to limit the present invention.

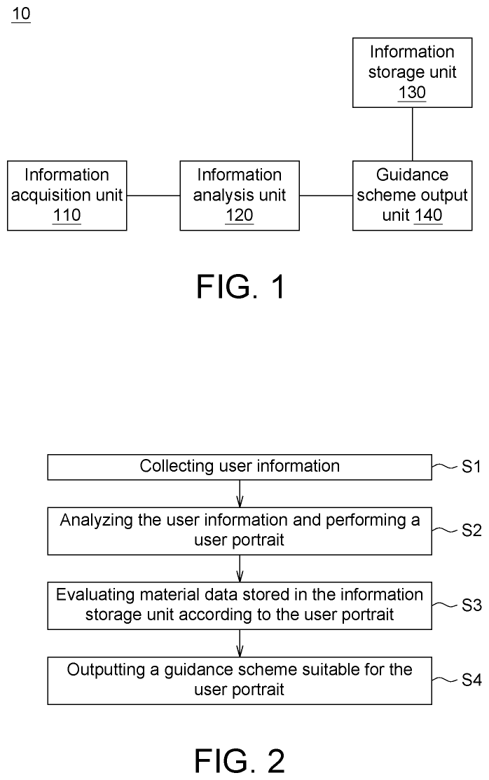

Referring to FIG. 1, FIG. 1 is a schematic diagram of multi-dimensional big health guidance system 10 based on a traditional Chinese medicine theory according to an embodiment of the invention. The multi-dimensional big health guidance system comprises an information acquisition unit 110, an information analysis unit 120, an information storage unit 130 and a guidance scheme output unit 140. The information acquisition unit 110 is connected to the information analysis unit 120, the information analysis unit 120 is connected to the guidance scheme output unit 140, and the guidance scheme output unit 140 also constructs information connection with the information storage unit 130.

More specifically, the information acquisition unit 110 is configured to collect user information and send the collected user information to the information analysis unit 120; the information analysis unit 120 analyzes and processes the user information to further draw a user portrait; and the guidance scheme output unit 140 obtains the user portrait from the information analysis unit 120 and performs relevance matching on the user portrait and the mass material data stored in the information storage unit 130 from multiple dimensions, so as to output a guidance scheme matching the user information.

In some embodiments, the information acquisition unit 110 further includes one or more of information acquisition modules, such as a data input module, an internet information acquisition module, a wearable device information receiving module, a face recognition module, a voice semantic recognition module and the like. The collected user information includes one or more of user basic information, disease information, physique information, physiological information, habitual information, behavior information, emotional information or environmental information. In more detail, the user basic information, such as gender, age, height, weight, family history, marital status and occupation, can be input through a human-machine interaction interface, or by using questionnaires to understand the user's health symptoms and living habits, or using a medical image scanner to identify physical examination data or medical image data to obtain the disease information, or using internet information to understand environmental information, such as the user's geographic environment, dietary preferences, season, climate, and weather etc., or through the information connection with the user's wearable device to obtain the user's heart rate, blood pressure, and exercise status, or through the facial recognition and speech semantic recognition devices to recognize the human's facial features, expression changes, voice changes, and semantics etc. The manner in which the user information is acquired by using the specific information acquisition module and devices is only illustrated, and the present invention is not limited thereto.

The information analysis unit 120 processes the user information, for example, the user information corresponding to a quantization value of one dimension, or splitting the same user information into a plurality of dimensions and quantizing the same user information into one value in each dimension, or dividing a part of different user information respectively as a quantization index of a certain dimension and aggregated into a quantized value in the dimension. Finally, the information analysis unit 120 may process the user information into a multi-dimensional coordinate system having a plurality of dimensions and a plurality of quantized value information in the dimensions, and simply speaking, the user portrait includes multi-dimensional information data. In some embodiments, the user portrait represents a location corresponding to the coordinate system according to user information. For a specific example, a user portrait is (a, b) where a location on which “a” is located represents the disease information, a location on which “b” is located represents the physique information; a value of “a” represents disease type and/or degree, and a value of “b” represents physique type and/or degree; or a user portrait is (c, d, e) where a location on which “c” is located represents the user health information, a value of “c” represents the health degree; a location on which “d” is located represents the geographic information at which the user is located, and a value of “d” represents the basic information of the location; a location on which “e” is located represents the time point information, and a value of “e” represents the time point at which the user is located. The above two-dimensional, three-dimensional coordinates are simply constructed by way of example to illustrate a construction form of a user portrait, and the actual user portrait may also be characterized by more-dimensional values. It should be noted that the above mentioned quantized value of a certain dimension (e.g. a\b\c\d\e value) is not limited to a numerical value or a binary code, a letter or any other content capable of expressing certain information. The user portrait is not limited to a multi-dimensional coordinate system, but also can be other information set that reflects information in multiple dimensions.

In another embodiment, the information analysis unit 120 incorporates an initial-order coordinate system, including (X₁, X₂, X₃, . . . , Xₙ) information of n dimensions (n≥2), the information analysis unit 120 processes the user information by one-to-one processing such as the user information R corresponding to the X₁ position, the quantized value is x1, or the user information S is split into such as X₂ and X₃ positions and the quantized values are x2 and x3, or at least a part of the user information corresponds to X₄ position and the quantized value is x4a, or at least part of the user information O also corresponds to X₄ position, and the quantified value is x4b, and x4a and x4b are aggregated into the value x4 where the X₄ position is located. Similarly, the information analysis unit 120 processes the user information one by one to generate the initial user portrait (x1, x2, x3, . . . , xn) (n≥2). In this embodiment, the information analysis unit 120 will further process the initial user portrait, extract the important feature information, and simplify it into a characteristic user portrait (y1, y2, . . . , ym) (m<n). In the step, the information analysis unit 120 will exclude some unimportant features, thereby simplifying the subsequent matching procedure.

The information storage unit 130 may be, for example, a built-in storage module or a cloud storage module including mass material data stored with, for example, one or more of food data, medicated diet formula, exercise methods, meridian massage, bath method, tea drink, and audio-visual material. The material data includes at least 847 food materials, efficacy and nutrition information, 600 diet curative effects and taboo information of multi-flavor traditional Chinese medicine, 262 taboo information of sports health care method, 80 intervention regulating projects of traditional Chinese medicine external treatment, 18 traditional Chinese medicine bath prescriptions, 400 Chinese medicine tea drinks and 60 audio-visual data. In some embodiments, the information storage unit 130 also stores correlation data between the material data and the information data representing the user portrait, and in a particular embodiment, the correlation data is based on the traditional Chinese medicine theory.

After the guidance scheme output unit 140 acquires user portrait drawn by the information analysis unit 120, relevance matching between the information data of a plurality of dimensions and mass material data stored in the information storage unit 130 may be performed, and a guidance scheme suitable for the user information is finally output. Alternatively, on the basis of the correlation data between the material data and the information data representing the user portrait, it is possible to match the user portrait to a suitable guidance scheme more quickly.

The guidance scheme output by the guidance scheme output unit 140 includes a catering scheme, a sports scheme, a leisure scheme and a beauty care scheme. For example, on the basis of basic attributes of all food materials, homology of medicine and food of traditional Chinese medicine and the principle of nature and taste returning to the meridian, the catering scheme of user's food matching for each meal is obtained. According to the user's physique, a sports regulating scheme that matches the season and time is obtained; according to the user's emotion and habits, a suitable leisure scheme is obtained; according to the user's skin property and environmental factors, a targeted beauty care scheme is obtained.

The embodiments of the invention disclose a multi-dimensional big health guidance system based on a traditional Chinese medicine theory and a multi-dimensional big health guidance method based on the traditional Chinese medicine theory. In summary, referring to FIG. 2, FIG. 2 is a schematic diagram of a multi-dimensional big health guidance method based on the traditional Chinese medicine theory according to an embodiment of the present invention. In this embodiment, a multi-dimensional big health guidance method based on the traditional Chinese medicine theory includes the following steps.

S1, collecting user information.

S2, analyzing the user information and performing a user portrait.

S3, evaluating material data stored in the information storage unit according to the user portrait.

S4, outputting a guidance scheme suitable for the user portrait.

In step S1, the user information is acquired by the information acquisition unit of the big health guidance system. The information acquisition unit includes a data input module, an internet information acquisition module, a wearable device information receiving module, a face recognition module, a voice semantic recognition module and the like. The user information includes user basic information, disease information, physique information, physiological information, habitual information, behavior information, emotional information, or environmental information

In step S2, the user information is converted into information data according to a plurality of dimensions by the information analysis unit, and the information data is three-dimensionally structured to form a user portrait. The three-dimensionally structure means that the information data and dimensional information are processed corresponding to a multi-dimensional coordinate system, or further processed corresponding to a characteristic multi-dimensional coordinate system.

In step S3, the information data of multiple dimensions is mainly relevance-matching the mass material data stored in the information storage unit by the guidance scheme output unit. In particular, such multi-dimensional relevance matching includes at least two methods: hierarchical matching or multi-dimensional synchronization matching. In step S4, outputting a guidance scheme suitable for the user portrait, i.e. outputting the final result of the hierarchical matching or the intersection set of the multi-dimensional synchronization matching result. A two-dimensional coordinate system (r, s) will be specifically described as follows.

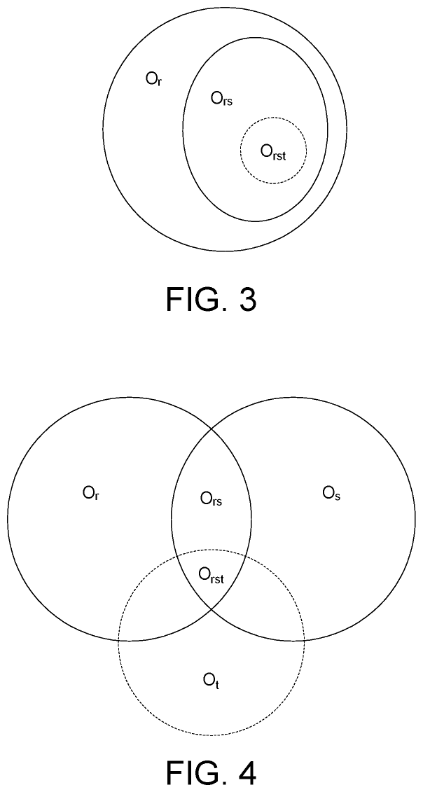

Referring to FIG. 3, FIG. 3 is a schematic diagram of a multi-dimensional hierarchical matching method according to an embodiment of the present invention. In this embodiment, the step S3 includes: analyzing the information data r from a first dimension, screening a first dimensional guidance scheme Oᵣ matching the first dimensional information data r from the information storage unit, and then analyzing the information data s from a second dimension to screen a second dimensional guidance scheme Oᵣₛ matching the second dimensional information data s from the first dimensional guidance scheme Oᵣ. In step S4, the hierarchical matching result Oᵣₛ is output. That is, the guidance scheme is finally output suitable for the user information. If the coordinate system then increases third dimensional information t, i.e. for the user portrait/feature user portrait (r, s, t), the analysis of step S3 is performed, then on the basis of the matching result Oᵣₛ at (r, s), the information data t is analyzed from a third dimension to screen a three-dimensional guidance scheme Oᵣₛₜ matching the third dimensional information data t from the two-dimensional guidance scheme Oᵣₛ. In step S4, the hierarchical matching result Oᵣₛₜ is output. That is, the guidance scheme is finally output suitable for the user information. Other multi-dimensional coordinate systems are analogy.

Referring to FIG. 4, FIG. 4 is a schematic diagram of a multi-dimensional hierarchical matching method according to an embodiment of the invention. In an embodiment of multi-dimensional synchronization matching, the step S3 includes analyzing the information data r from a first dimension to screen a first dimensional guidance scheme Oᵣ matching the first dimensional information data from the information storage unit; and synchronously analyzing the information data s from a second dimension to screen a second dimensional guidance scheme Oₛ matching the second dimensional information data s from the information storage unit, wherein synchronization matching does not represent simultaneous operation, simply represents that both of information data are analyzed in the same step. A two-dimensional guidance scheme Oᵣₛ is obtained from the intersection set of the first dimensional guidance scheme Oᵣ and the second dimensional guidance scheme Oₛ. That is, the guidance scheme Oᵣₛ from Oᵣ and Oₛ is determined and screened synchronously. In step S4, the multi-dimensional synchronous matching result Oᵣₛ is output. That is, the guidance scheme is finally output suitable for the user information. If the coordinate system re-increases third dimension information t, i.e. for the user portrait or feature user portrait (r, s, t), the information data t is analyzed from a third dimension to screen a third dimensional guidance scheme Oₜ that matches the third dimensional information data t from the information storage unit. A three-dimensional guidance scheme Oᵣₛₜ matching the third dimensional information data t from the information storage unit is obtained from the intersection set of guidance schemes Oᵣ\Oₛ\Oₜ. In step S4, namely the multi-dimensional synchronous matching result Oᵣₛₜ is output. That is, the guidance scheme is finally output suitable for the user information. Other multi-dimensional coordinate systems are analogy.

In one embodiment of the invention, the information acquisition unit collects user information and the user information is processed into information data of at least two dimensions by the information analysis unit. At least one dimensional information is related to the user's personal physique or behavior and habits, referred to as personal factors; and at least another dimensional information is related to the user's environment, referred to as environmental factors. In particular, personal factors include user's occupation, disease, physique, religious, and emotion etc., and environmental factors include geographical, climate, weather, season, time point, and the like.

The traditional Chinese medicine believes that the human body itself is an organic whole and should remain unity with the natural environment. By taking the diet as an example, the traditional Chinese medicine nutrition takes heaven, earth and human as a reference system, and it is considered that foods corresponding to the current season are best eaten. On one hand, the foods to be drunk or eaten by people have to speak the time of production, the time of consumption, and the amount of consumed food. We must eat in a modest manner and in accordance with the weather, which means that we must apply the season. In addition to the season, we also “apply” the land, “south rice north wheat”, “east-west nap”, so-called one-water-soil cultivation, our people's physique is created by the land where we live, so that the local food is most suitable for people to eat.

The foods can not only provide essential nutrients to the human body, but can also maintain and promote the health of the body, reduce the risk of diseases and prevent the occurrence of diseases. As so-called “medicine and diet are homology” in the traditional Chinese medicine theory, the physique of each person is different, the nature of each type of the foods is different, and reasonable matching is needed for each person according to the specific situation. For example, red dates belong to invigorating drugs, and have the efficacy of moistening lung, relieving cough, nourishing five internal organs and treating the deficiency. People with poor gastrointestinal function or digestion and absorption are suitable for common-eating red dates to improve illness state, gain physical strength and help restore normal detoxification of intestines and stomach. The red dates are high in sugar content, can generate a lot of heat and can be eaten by people with coldness, and can change the cold state of the physique. The lotus seeds are sweet and astringent in nature, acting on heart, spleen, kidney and having the functions of heart nourishing, kidney nourishing, inhibition of myocardial contractility, heart rate reduction, coronary artery expansion, blood vessels relaxing, blood pressure reduction, spleen invigorating, intestines nourishing, anti-aging and prolonged service life. When the lotus seeds are used as dietary therapy, the lotus seed heart is generally not discarded, and the lotus seed heart is bitter, and has the effects of clearing heart heat, fixing essence, calming nerves and strengthening heart.

Take another simple example about food, such as a meal ordering application (APP), the information analysis unit 110 may selectively obtain user information, such as the user's identity, the age, the ordering habit history, and combine seasonal information to obtain merchant information near the user's location, such as including information about specialty restaurants, delivery speeds, and consumption levels etc. The information analysis unit 120 can comprehensively analyze the above data, and perform matching from the information storage unit 130 that stores all merchant catering schemes, and finally the guidance scheme output unit 140 provides a catering scheme recommendation that may be of interest to this order, or provides a plurality of reference schemes from different dimensions, such as the most season food catering scheme, the most rapid distribution catering scheme and the most common food catering scheme.

In this embodiment, the big health guidance scheme obtained by personal factors must be adapted to the personal condition of the user to be consistent with the “man”, and the most recent agricultural solar term and the weather change condition can be acquired through weather APP, and then understand season food information to be consistent with the “nature”; the geographical and climate information of the position where the user is located can be obtained through GPS positioning, and then understand the feature of a local diet to be consistent with the “land”. The concept of “unity of man and nature” or “the harmony between man and nature” means to conform to the diet requirements of the human body due to different solar terms in four seasons, and the inner essence is harmonious with the external environment, so that our life can be best maintained. It is to be noted that, although in the foregoing discussion, the traditional Chinese medicine concept to be consistent with the “nature”, the “land”, and the “man” is particularly emphasized, the guidance scheme of the present invention is not limited thereto. According to the aforementioned schemes provided by the information analysis unit for processing user information, some unimportant features will be excluded to simplify the matching procedure.

Take another specific example of beauty care, such as a skin care APP, the information acquisition unit 110 can selectively collect user information, such as the user's identity, age, physique, skin property, catering work habit and the like, and combine information such as geographic environment, climate and season, and the information analysis unit 120 can comprehensively analyze the data and perform matching from the information storage unit 130 that stores mass material data. Finally, the guidance scheme output unit 140 provides a beauty care scheme matching the user's identity and age, which is suitable for the user's physique and skin property and a catering, sports, health care, skin maintenance matching the local environment and climate.

Of course, the above-mentioned embodiments are merely illustrative of the central idea of the present invention, and in other embodiments, when the environmental factor is not significant, the user information can be quantized and become information data of at least two dimensions, and the two-dimensional information data is related to personal factors of the user, which are different aspects of personal factors, such as different combinations of disease and physique, age and emotion, etc.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A big health guidance system based on a traditional Chinese medicine theory, comprising:
an information acquisition unit used for collecting user information, the information acquisition unit at least comprises one or more of a data input module, an internet information acquisition module, a wearable device information receiving module, a face recognition module and a voice semantic recognition module;
an information analysis unit used for analyzing the user information and performing a user portrait, and the user information comprises at least one or more of basic information, disease information, physique information, physiological information, habitual information, behavior information, emotional information and environmental information, wherein at least a first dimensional information data of the user information is related to user's personal factors and a second dimensional information data of the user information is related to user's environment, the first dimensional information data is obtained from one or more of a meal ordering application (APP), a sports or a health class APP and a beauty treatment APP, the second dimensional information data is obtained from the user's geographic environment, dietary preferences, season, climate, and weather or time point information, wherein the information analysis unit processes the user information into the user portrait having a plurality of dimensions and a plurality of quantized value information in the dimensions;
an information storage unit used for storing material data related to user's basic necessities, and the material data comprises one or more of food material data, a medicated diet formula, an exercise method, a meridian massage, a bath method, a tea drink and an audio-visual material;
a guidance scheme output unit used for performing a relevance matching on the user portrait and the material data and outputting a guidance scheme suitable for the user portrait based on the traditional Chinese medicine theory.

2. The system according to claim 1, wherein the user portrait comprises multi-dimensional information data.

3. The system according to claim 2, wherein the information storage unit further stores correlation data between the material data and the user information data, and the correlation data is based on the traditional Chinese medicine theory.

4. The system according to claim 1, wherein the guidance scheme comprises a catering scheme, a sports scheme, a leisure scheme and a beauty care scheme.

5. The system according to claim 4, wherein the catering scheme is applied to the meal ordering application (APP); the sports scheme or the leisure scheme is applied to the sports or health class APP; and the beauty care scheme is applied to the beauty treatment APP.

6. A multi-dimensional big health guidance method based on a traditional Chinese medicine theory, comprising following steps:
S1, collecting user information, including one or more information of a user input, an image scanning, a wearing device information receiving, an internet access, a face recognition, a speech semantic recognition;
S2, analyzing the user information and performing a user portrait by converting the user information into information data according to a plurality of dimensions, wherein at least a first dimensional information data of the user information is related to user's personal factors and a second dimensional information data of the user information is related to user's environment factors, the first dimensional information data is obtained from one or more of a meal ordering application (APP), a sports or a health class APP and a beauty treatment APP, the second dimensional information data is obtained from the user's geographic environment, dietary preferences, season, climate, and weather or time point information, wherein the user information is processed into the user portrait having the plurality of dimensions and a plurality of quantized value information in the dimensions;
S3, evaluating material data stored in the information storage unit according to the user portrait; and
S4, outputting a guidance scheme suitable for the user portrait.

7. The method according to claim 6, wherein the step S3 further comprises: analyzing the information data from a first dimension to screen a first dimensional guidance scheme matching the first dimensional information data from the information storage unit; and then analyzing the information data from a second dimension to screen a two-dimensional guidance scheme matching the second dimensional information data from the first dimensional guidance scheme.

8. The method according to claim 6, wherein the step S3 further comprises: analyzing the information data from a first dimension to screen a first dimensional guidance scheme matching the first dimensional information data from the information storage unit; and analyzing the information data from a second dimension to screen a second dimensional guidance scheme matching the second dimensional information data from the information storage unit; and analyzing the first dimensional guidance scheme and the second dimensional guidance scheme and screening an intersection set as a two-dimensional guidance scheme.

9. The method according to claim 7, wherein the step S3 further comprises: analyzing the information data from a third dimension to screen a three-dimensional guidance scheme matching the third dimensional information data from the two-dimensional guidance scheme.

10. The method according to claim 8, wherein the step S3 further comprises: analyzing the information data from a third dimension to screen a three-dimensional guidance scheme matching the third dimensional information data from the two-dimensional guidance scheme.

11. The method according to claim 7, wherein the step S3 further comprises: synchronously analyzing the information data from a third dimension to screen a third dimensional guidance scheme matching the third dimensional information data from the information storage unit, analyzing the two-dimensional guidance scheme and the third dimensional guidance scheme and screening an intersection set as a three-dimensional guidance scheme.

12. The method according to claim 8, wherein the step S3 further comprises: synchronously analyzing the information data from a third dimension to screen a third dimensional guidance scheme matching the third dimensional information data from the information storage unit, analyzing the two-dimensional guidance scheme and the third dimensional guidance scheme and screening an intersection set as a three-dimensional guidance scheme.
